(19) United States
(12) Patent Application Publication
(10) Pub. No.: US 2020/0216916 A1
(43) Pub. Date: Jul. 9, 2020

(54) METHOD FOR ESTIMATING ADDITIVE AND DOMINANT GENETIC EFFECTS OF SINGLE METHYLATION POLYMORPHISMS (SMPS) ON QUANTITATIVE TRAITS

(71) Applicant: Beijing Forestry University, Beijing (CN)

(72) Inventors: Deqiang ZHANG, Beijing (CN); Liang XIAO, Beijing (CN); Qingzhang DU, Beijing (CN); Mingyang QUAN, Beijing (CN); Wenjie LU, Beijing (CN); Panfei CHEN, Beijing (CN)

(73) Assignee: Beijing Forestry University, Beijing (CN)

(21) Appl. No.: 16/585,993

(22) Filed: Sep. 27, 2019

(30) Foreign Application Priority Data
Jan. 3, 2019 (CN) 201910005389.1

(51) Int. Cl. C12Q 1/6895; C12Q 1/6827; G16B 40/10; G16B 20/20; C40B 40/06

(52) U.S. Cl. CPC C12Q 1/6895; C12Q 1/6827; G16B 40/10; C12Q 2600/154; C40B 40/06; C12Q 2600/13; G16B 20/20

(57) ABSTRACT

The present invention relates to the field of plant molecular breeding, and provides methods for estimating additive and dominant genetic effects of single methylation polymorphisms (SMPs) on quantitative traits. The method comprises the following steps: 1) collecting samples and measuring phenotype in a natural population, and extracting genomic DNA from the samples; 2) constructing MethylC-seq libraries using the sample genomic DNA, and sequencing; 3) identifying the SMPs from the DNA methylation sequencing reads, and performing genotyping; and 4) performing epigenome-wide association study on the SMPs and the phenotypic data using a Mixed Linear Model (MLM), identifying SMPs that are significantly associated with the phenotype, and estimating the additive and dominant genetic effects. The method can provide a new technical guidance for gene marker-assisted breeding, and has important theoretical and breeding values.

### CROSS-REFERENCE TO RELATED APPLICATIONS AND CLAIM TO PRIORITY

This application claims priority to Chinese application number 201910005389.1, filed Jan. 3, 2019, entitled METHOD FOR DETECTING ADDITIVE AND DOMINANT GENETIC EFFECTS OF DNA METHYLATION SITES ON QUANTITATIVE TRAITS AND USE THEREOF, which is incorporated herein by reference in its entirety.

### FIELD OF THE INVENTION

The present invention relates to the field of plant molecular breeding, and in particular, to methods for estimating additive and dominant genetic effects of single methylation polymorphisms (SMPs) on quantitative traits.

### BACKGROUND OF THE INVENTION

DNA methylation is a covalent base modification of nuclear genomes that is accurately inherited through both mitosis and meiosis, which is present in the CG, CHG and CHH contexts (where H=A, C or T). Similar to the SNP generated by spontaneous mutations in DNA sequence, due to the low fidelity of DNA methyltransferase in the genome, errors in the maintenance of the methylation status result in the accumulation of single methylation polymorphisms (SMPs) over an evolutionary timescale, and about 6-25% of cytosines are methylated in higher plant genomes. The natural SMPs with different epialleles can exhibit distinct phenotypes. For example, due to increasing methylation density of Lcyc genes in Linaria vulgaris, the fundamental symmetry of the flower has changed from bilateral to radial, indicating that DNA methylation may play a significant role in that phenotypic variation, and SMPs can be as an important marker to explore the epigenetic mechanism of complex traits.

Many traits that are important for adaptability and growth of plants are complex quantitative traits, affected by multiple genes in different biological pathways. In addition, dissection of genetic architecture reveals the importance of additive and dominant effects of gene in complex traits. The additive effect represents the breeding value of the traits and is the main component of the phenotypic value of the traits. The dominant effect is the effect produced by the interaction between allelic loci, i.e., the difference of a genotype value (G) and an additive effect value (D). Although previous studies have demonstrated the regulatory role of SMPs in plant complex traits, the additive and dominant genetic effects of SMPs, which indicate the breeding value, have not been estimated.

### SUMMARY OF THE INVENTION

In view of the above, an objective of the present invention is to provide methods for estimating additive and dominant genetic effects of single methylation polymorphisms (SMPs) on quantitative traits. The methods can scientifically and accurately detect the additive and dominant genetic effects on quantitative traits, and provide new marker resources for marker-assisted breeding, which has important theoretical and breeding values.

To achieve the above purpose, the present invention provides the following technical solutions.

A method for estimating additive and dominant genetic effects of single methylation polymorphisms (SMPs) on quantitative traits includes the following steps:

1) collecting the samples of different individuals in a natural population at the same stage and in the same tissue, and isolating the genomic DNA of each sample; measuring the phenotypic data from the individuals in the natural population;

2) constructing MethylC-seq libraries using the genomic DNA of each sample in step 1), and performing paired-end sequence to obtain DNA methylation sequencing reads;

3) identifying single methylation polymorphisms (SMPs) from the DNA methylation sequencing reads, and performing genotyping according to the methylation support rate (MSR) of the DNA methylation sites in each individual, which is calculated by the formula:

$$DNA methylation support rate ( MSR ) = \frac{methylated reads}{methylated reads + unmethylated reads}$$

if MSR of the site is >0.7, the genotyping is homozygous methylated site (M:M); if MSR of the site is between 0.3 and 0.7, the genotyping is heterozygous site (U:M); and if MSR of the site is <0.3, the genotyping is homozygous unmethylated site (U:U);

4) performing epigenome-wide association study on SMPs obtained in step 3) and the phenotypic data in step 1) by Mixed Linear Model (MLM), and identifying SMPs that are significantly associated with the phenotype;

5) estimating the additive and dominant genetic effects of the significantly associated SMPs using the Tassel 5.0 software package.

Preferably, a threshold for the identifying the significantly associated SMPs in step 4) is P<1/n (Bonferroni correction), where n is the number of SMPs.

Preferably, software for the identifying SMPs, and performing genotyping according to the methylation support rate of the DNA methylation sites in step 3) is the Bismark software.

Preferably, the DNA methylation sequencing in step 2) is paired-end sequencing with a read length of 125 bp and a depth of 30×; and the sequencing is performed by the Illumina Hiseq 2000/2500 platform.

Preferably, the samples are from perennial woody plants.

Preferably, the phenotypic data includes leaf area and stomatal conductance.

The present invention provides a method for plant molecular breeding.

The advantageous effects of the present invention: the methods provided by the present invention first considers the additive and dominant genetic effects of SMPs, while analyzing the epigenetic variation mechanism of DNA methylation on complex quantitative traits. The methods provide a scientific theoretical basis for the efficient analysis of the epigenetic variation mechanism of complex quantitative traits of perennial woody plants, and a new technical guidance for gene marker-assisted breeding, which has important theoretical and technical values.

### BRIEF DESCRIPTION OF THE DRAWINGS

FIG. 1 is a Manhattan plot showing the results of the epigenome-wide association study of the leaf area trait in Example 1; and

FIG. 2 is a Manhattan plot showing the results of the epigenome-wide association study of the stomatal conductance trait in Example 2.

### DETAILED DESCRIPTION OF THE PREFERRED EMBODIMENTS

The present invention provides methods for detecting additive and dominant genetic effects of SMPs on quantitative traits, including the following steps:

1) collecting samples of different individuals in natural population at the same stage and in same tissue, isolating the genomic DNA of each sample, and measuring the phenotypic data from the individuals the natural population;

2) constructing MethylC-seq libraries using the genomic DNA of each sample in step 1), and performing paired-end sequence to obtain DNA methylation sequencing reads;

3) identifying genome-wide single methylation polymorphisms (SMPs) from the DNA methylation sequencing reads, and performing genotyping according to the methylation support rate (MSR) of the DNA methylation sites in each individual, which is calculated by the formula:

$$DNA methylation support rate ( MSR ) = \frac{methylated reads}{methylated reads + unmethylated reads}$$

if MSR of the site is >0.7, the genotyping is homozygous methylated site (M:M); if MSR of the site is between 0.3 and 0.7, the genotyping is heterozygous site (U:M); and if MSR of the site is <0.3, the genotyping is homozygous unmethylated site (U:U);

4) performing epigenome-wide association study on the SMPs obtained in step 3) and the phenotypic data in step 1) by Mixed Linear Model (MLM), and identifying SMPs that are significantly associated with the phenotype;

5) analyzing the additive and dominant genetic effects of the significantly associated SMPs by the Tassel 5.0 software package.

In the present invention, the samples of different individuals in the natural population are collecting at the same stage and in same tissue; and the phenotypic data are measured from the natural population. The present invention has no particular limitation on the species of the sample. The sample is preferably a plant, and more preferably, a perennial woody plant. In the specific implementation of the present invention, the sample is preferably from Populus tomentosa. In the present invention, the tissue is preferably a leaf. The present invention preferably collects the leaf tissues of different individuals at the same time in the same growth environment, so as to eliminate the influence of environmental effects, growth states and tissue-specificity on DNA methylation sites, thereby identifying SMPs to resolve the additive and dominant genetic effects of SMPs. The present invention has no particular limitation on the phenotypic traits, but a phenotype having practical application significance is preferred. In the specific implementation of the present invention, the phenotype is preferably leaf area and/or stomatal conductance. The present invention has no particular limitation on the phenotypic trait detection method; and a conventional phenotypic trait detection method may be employed.

The present invention isolates the genomic DNA from each sample to obtain genomic DNA. The present invention has no particular limitation on the genomic DNA isolation method; and a conventional genomic DNA extraction method may be used. Preferably, a plant genomic DNA extraction kit is used. Specifically, a DNeasy Plant Mini Kit (Qiagen China, Shanghai, China) is used for extraction. The QiAGEN DNeasy Plant Mini Kit provides rapid and easy purification of the genomic DNA via a gel membrane-based spin column. The genomic DNA isolated from the samples described in the present invention within a specific stage and specific tissue is used to facilitate genotyping of the DNA methylation sites. After extracting the sample genomic DNA, Nanodrop is used to detect an OD260/OD280 ratio of each DNA sample to determine the purity of the DNA sample. OD260/OD280≈1.8 indicates high DNA purity. OD260/OD280 >1.9 indicates RNA contamination. OD260/OD280<1.6 indicates contamination with protein and phenol. After the purity and integrity detection, the present invention preferably further includes: detecting the concentration of the genomic DNA by the Qubit 2.0 Flurometer (Life Technologies, CA, USA).

The present invention constructs MethylC-seq libraries using each genomic DNA of the sample. In the specific implementation of the present invention, the method for constructing the MethylC-seq libraries specifically includes the following steps: 2.1) randomly fragmenting the genomic DNA to 200-300 bp; 2.2) performing terminal modification on the DNA fragment by adding a tail A, and ligating a sequencing adapter; and 2.3) performing PCR amplification after twice treating the ligated DNA fragment with bisulfite. In the present invention, the all cytosines in the sequencing adapter are methylated, and the function of the ligated sequence adapter is to provide sequence information for primers required for the sequencing by amplification process. In the present invention, after the bisulfite treatment, the un-methylated C becomes U (which becomes T after PCR amplification), and the methylated C remains unchanged. In the present invention, the bisulfite treatment is preferably carried out using an EZ DNA Methylation Gold Kit (Zymo Research, Murphy Ave., Irvine, Calif., U.S.A.). The present invention has no particular limitation on the method for constructing the MethylC-seq library. A conventional method for constructing a MethylC-seq library in the art may be used; or the construction of the MethylC-seq library may be entrusted to a biological sequencing company.

After obtaining the MethylC-seq library, the present invention performs DNA methylation sequencing to obtain the DNA methylation sequencing data. In the present invention, the DNA methylation sequencing is preferably paired-end sequencing with a read length of 125 bp and a depth of 30×, and the sequencing is preferably performed using an Illumina Hiseq 2000/2500 platform. In the specific implementation of the present invention, the DNA methylation sequencing is preferably entrusted to Beijing Novogene Biological Information Technology Co., Ltd.

After DNA methylation sequencing, the present invention identifies genome-wide SMPs from the DNA methylation sequencing reads, and performs genotyping according to the methylation support rate (MSR) of the DNA methylation sites in each individual, which calculated by the formula:

$$DNA methylation support rate ( MSR ) = \frac{methylated reads}{methylated reads + unmethylated reads}$$

if MSR of the site is >0.7, the genotyping is homozygous methylated site (M:M); if MSR of the site is between 0.3 and 0.7, the genotyping is heterozygous site (U:M); and if MSR of the site is <0.3, the genotyping is homozygous unmethylated site (U:U);

In the present invention, the foregoing operation is preferably performed using the Bismark software. The genotyping data of the SMPs obtained by the present invention can be used to perform epigenome-wide association study of SMPs-phenotype to explore the genetic effects of DNA methylation.

After obtaining the genotyping data of the SMPs, the present invention performs epigenome-wide association study on SMPs and the phenotypic data by using a Mixed Linear Model (MLM), and identifies the SMPs significantly associated with the phenotype. In the present invention, a threshold for the identifying the significantly associated DNA methylation sites is P<1/n (Bonferroni correction), where n is the number of SMPs. In the specific implementation of the present invention, the MLM module is preferably selected in the Tassel 5.0 software package, and the population structure and kinship matrix are set as covariates.

After obtaining the significantly associated SMPs, the present invention analyzes the additive and dominant genetic effects of the significantly associated SMPs by the Tassel 5.0 software package.

The present invention also provides use of the foregoing method in plant molecular breeding, and preferably used in plant molecular assisted breeding. The present invention has no particular limitation on the specific method of application.

The technical solution provided by the present invention are described below in detail with reference to examples. However, the examples should not be construed as limiting the protection scope of the present invention.

### Example 1

Specific operation steps are as follows:

Step 1): The natural population is of 5-year-old, 300 Populus tomentosa genotypic individuals planted in Guanxian County, Shandong, China. The functional leaves (the fourth to sixth leaves from the top of the stem) are collected from 9:00 to 11:00 AM, and in order to prevent changes in its DNA methylation pattern, and are immediately frozen in liquid nitrogen (−196° C.) after collection.

Step 2): the genomic DNA of the leaf samples are isolated using DNeasy Plant Mini Kit (Qiagen China, Shanghai, China).

After the foregoing steps are completed, the genomic DNA can be further detected, specifically: 2.1: the degree of degradation of the DNA sample and the RNA contamination are determined by agarose gel electrophoresis; 2.2: the OD260/OD280 ratio of each DNA sample is detected using Nanodrop to determine the purity of the DNA sample; and 2.3: the concentration of each DNA sample is accurately quantified using Qubit2.0 Flurometer (Life Technologies, CA, USA).

Then, the methods of performing bisulfite sequencing on the extracted genomic DNA and constructing the bisulfite-treated DNA library based on the genomic DNA in step 3) uses a conventional technical method, and the specific implementation of the present invention is as follows:

Step 3.1: the genomic DNA is randomly fragment to 200-300 bp by using Covaris S220.

Step 3.2: end repairing and tail A addition are performed on the DNA fragments, using the sequencing adapters in which all cytosines are methylated, the purpose of which is to provide sequence information for the primers required for PCR amplification.

Step 3.3: the DNA fragments in step 3.2 are twice treat with bisulfite, and after the bisulfite treatment, the C which is not methylated becomes U (which becomes T after PCR amplification), and the methylated C remains unchanged. Specifically, the bisulfite treatment is carried out using an EZ DNA Methylation Gold Kit (Zymo Research, Murphy Ave., Irvine, Calif., U.S.A.).

Step 3.4: the bisulfite-treated DNA fragments in step 3.3 are subjected to PCR amplification to construct a MethylC-seq library.

Step 3.5: sequencing is performed on MethylC-seq library.

The DNA isolation, MethylC-seq library construction, and sequencing were performed on Beijing Novogene Biological Information Technology Co., Ltd.

Step 4): identifying DNA methylation sites according to a sequencing reads of each sample, and performing genotyping on the SMPs. The sequencing reads of each sample were aligned to the Populus tomentosa reference genome using the Bismark and the Bowtie2 software, with default parameters to identify the SMPs. The methylation support rate of each DNA methylation site is calculated for genotyping. Specifically, the methylation support rate (MSR) of the DNA methylation sites in each individual, which calculated by the formula:

$$DNA methylation support rate ( MSR ) = \frac{methylated reads}{methylated reads + unmethylated reads}$$

if MSR of the site is >0.7, the genotyping is homozygous methylated site (M:M); if MSR of the site is between 0.3 and 0.7, the genotyping is heterozygous site (U:M); and if MSR of the site is <0.3, the genotyping is homozygous unmethylated site (U:U);

Step 5) Measurement of leaf area traits. The functional leaves (the fourth to sixth leaves from the top of the stem) are collected at the same time as the leaf samples for extracting the genomic DNA. Then, the functional leaves of each individuals were used to measure the leaf area by CI-202 portable laser leaf area meter (CID Bio-Science, Inc., Camas, Wash., USA). The leaf area phenotypic value is shown in Table 1.

TABLE 1
Leaf area of 300 genotypic individuals of a natural
population of Populus tomentosa (unit: cm²)

| Individual | Leaf |
|---|---|
| No. | area |
| P1 | 49.083 |
| P2 | 59.855 |
| P3 | 49.930 |
| P4 | 36.623 |
| P5 | 40.853 |
| P6 | 38.860 |
| P7 | 40.840 |
| P8 | 69.623 |
| P9 | 50.240 |
| P10 | 33.293 |
| P11 | 65.273 |
| P12 | 50.123 |
| P13 | 68.125 |
| P14 | 40.953 |
| P15 | 45.693 |
| P16 | 43.947 |
| P17 | 40.073 |
| P18 | 49.123 |
| P19 | 61.123 |
| P20 | 52.210 |
| P21 | 31.343 |
| P22 | 46.910 |
| P23 | 37.695 |
| P24 | 38.763 |
| P25 | 48.915 |
| P26 | 40.588 |
| P27 | 41.583 |
| P28 | 51.040 |
| P29 | 40.373 |
| P30 | 45.067 |
| P31 | 37.533 |
| P32 | 47.357 |
| P33 | 60.853 |
| P34 | 58.697 |
| P35 | 48.353 |
| P36 | 43.053 |
| P37 | 49.500 |
| P38 | 37.577 |
| P39 | 47.467 |
| P40 | 56.023 |
| P41 | 52.110 |
| P42 | 54.677 |
| P43 | 51.950 |
| P44 | 36.813 |
| P45 | 53.353 |
| P46 | 41.260 |
| P47 | 79.670 |
| P48 | 35.470 |
| P49 | 53.010 |
| P50 | 43.687 |
| P51 | 44.827 |
| P52 | 58.093 |
| P53 | 35.393 |
| P54 | 43.487 |
| P55 | 61.603 |
| P56 | 70.720 |
| P57 | 35.943 |
| P58 | 54.493 |
| P59 | 61.335 |
| P60 | 46.500 |
| P61 | 39.470 |
| P62 | 73.667 |
| P63 | 37.892 |
| P64 | 54.569 |
| P65 | 71.414 |
| P66 | 76.283 |
| P67 | 34.955 |
| P68 | 56.178 |
| P69 | 34.529 |
| P70 | 53.192 |
| P71 | 52.071 |
| P72 | 73.927 |
| P73 | 42.210 |
| P74 | 39.881 |
| P75 | 54.557 |
| P76 | 70.088 |
| P77 | 54.795 |
| P78 | 39.476 |
| P79 | 55.622 |
| P80 | 59.773 |
| P81 | 66.672 |
| P82 | 37.155 |
| P83 | 38.168 |
| P84 | 44.874 |
| P85 | 64.770 |
| P86 | 71.582 |
| P87 | 66.887 |
| P88 | 76.834 |
| P89 | 45.763 |
| P90 | 74.009 |
| P91 | 48.508 |
| P92 | 75.425 |
| P93 | 34.930 |
| P94 | 55.451 |
| P95 | 40.035 |
| P96 | 44.023 |
| P97 | 35.823 |
| P98 | 54.938 |
| P99 | 68.346 |
| P100 | 57.539 |
| P101 | 28.577 |
| P102 | 42.988 |
| P103 | 46.291 |
| P104 | 49.900 |
| P105 | 62.410 |
| P106 | 39.532 |
| P107 | 70.836 |
| P108 | 30.866 |
| P109 | 31.078 |
| P110 | 39.121 |
| P111 | 61.967 |
| P112 | 37.722 |
| P113 | 29.301 |
| P114 | 66.277 |
| P115 | 54.727 |
| P116 | 33.596 |
| P117 | 73.800 |
| P118 | 55.943 |
| P119 | 34.167 |
| P120 | 73.484 |
| P121 | 38.289 |
| P122 | 76.656 |
| P123 | 75.219 |
| P124 | 33.297 |
| P125 | 49.464 |
| P126 | 68.489 |
| P127 | 66.641 |
| P128 | 29.645 |
| P129 | 74.485 |
| P130 | 28.387 |
| P131 | 54.633 |
| P132 | 59.134 |
| P133 | 62.161 |
| P134 | 45.621 |
| P135 | 41.156 |
| P136 | 36.315 |
| P137 | 50.044 |
| P138 | 48.783 |
| P139 | 57.555 |
| P140 | 39.324 |
| P141 | 69.668 |
| P142 | 28.293 |
| P143 | 55.258 |
| P144 | 71.853 |
| P145 | 29.790 |
| P146 | 41.682 |
| P147 | 63.049 |
| P148 | 73.299 |
| P149 | 44.750 |
| P150 | 34.424 |
| P151 | 49.343 |
| P152 | 61.850 |
| P153 | 48.575 |
| P154 | 77.912 |
| P155 | 43.120 |
| P156 | 55.207 |
| P157 | 61.314 |
| P158 | 61.479 |
| P159 | 41.501 |
| P160 | 35.072 |
| P161 | 45.791 |
| P162 | 30.921 |
| P163 | 32.816 |
| P164 | 62.476 |
| P165 | 75.361 |
| P166 | 67.696 |
| P167 | 30.662 |
| P168 | 60.338 |
| P169 | 53.910 |
| P170 | 31.342 |
| P171 | 67.656 |
| P172 | 53.879 |
| P173 | 51.972 |
| P174 | 77.709 |
| P175 | 53.074 |
| P176 | 37.112 |
| P177 | 77.032 |
| P178 | 33.794 |
| P179 | 58.133 |
| P180 | 44.387 |
| P181 | 32.296 |
| P182 | 28.201 |
| P183 | 59.196 |
| P184 | 69.913 |
| P185 | 34.461 |
| P186 | 73.376 |
| P187 | 36.657 |
| P188 | 28.777 |
| P189 | 45.385 |
| P190 | 54.075 |
| P191 | 73.212 |
| P192 | 76.185 |
| P193 | 31.726 |
| P194 | 53.727 |
| P195 | 68.299 |
| P196 | 72.902 |
| P197 | 34.605 |
| P198 | 60.115 |
| P199 | 28.971 |
| P200 | 46.561 |
| P201 | 39.706 |
| P202 | 64.099 |
| P203 | 58.639 |
| P204 | 55.944 |
| P205 | 67.451 |
| P206 | 47.302 |
| P207 | 39.418 |
| P208 | 48.549 |
| P209 | 58.114 |
| P210 | 36.017 |
| P211 | 48.257 |
| P212 | 55.182 |
| P213 | 74.486 |
| P214 | 56.220 |
| P215 | 28.831 |
| P216 | 48.770 |
| P217 | 44.003 |
| P218 | 32.474 |
| P219 | 28.426 |
| P220 | 54.987 |
| P221 | 51.716 |
| P222 | 60.996 |
| P223 | 45.842 |
| P224 | 69.373 |
| P225 | 70.203 |
| P226 | 54.424 |
| P227 | 54.551 |
| P228 | 57.263 |
| P229 | 31.684 |
| P230 | 33.353 |
| P231 | 59.161 |
| P232 | 36.854 |
| P233 | 71.878 |
| P234 | 63.735 |
| P235 | 72.703 |
| P236 | 63.190 |
| P237 | 43.626 |
| P238 | 45.447 |
| P239 | 63.674 |
| P240 | 61.973 |
| P241 | 49.860 |
| P242 | 40.573 |
| P243 | 47.432 |
| P244 | 46.447 |
| P245 | 37.605 |
| P246 | 43.497 |
| P247 | 29.440 |
| P248 | 30.064 |
| P249 | 47.393 |
| P250 | 46.697 |
| P251 | 31.023 |
| P252 | 52.193 |
| P253 | 63.787 |
| P254 | 48.363 |
| P255 | 37.305 |
| P256 | 43.833 |
| P257 | 59.904 |
| P258 | 63.976 |
| P259 | 75.217 |
| P260 | 67.104 |
| P261 | 48.533 |
| P262 | 70.309 |
| P263 | 36.488 |
| P264 | 29.788 |
| P265 | 32.623 |
| P266 | 35.577 |
| P267 | 47.400 |
| P268 | 66.821 |
| P269 | 30.767 |
| P270 | 48.007 |
| P271 | 34.967 |
| P272 | 45.603 |
| P273 | 41.774 |
| P274 | 64.766 |
| P275 | 61.117 |
| P276 | 48.990 |
| P277 | 35.583 |
| P278 | 47.577 |
| P279 | 70.887 |
| P280 | 67.749 |
| P281 | 30.258 |
| P282 | 39.828 |
| P283 | 59.357 |
| P284 | 55.322 |
| P285 | 40.718 |
| P286 | 76.666 |
| P287 | 44.021 |
| P288 | 41.988 |
| P289 | 59.963 |
| P290 | 32.149 |
| P291 | 65.665 |
| P292 | 49.786 |
| P293 | 69.942 |
| P294 | 71.353 |
| P295 | 69.399 |
| P296 | 77.248 |
| P297 | 40.207 |
| P298 | 68.124 |
| P299 | 55.493 |
| P300 | 35.035 |

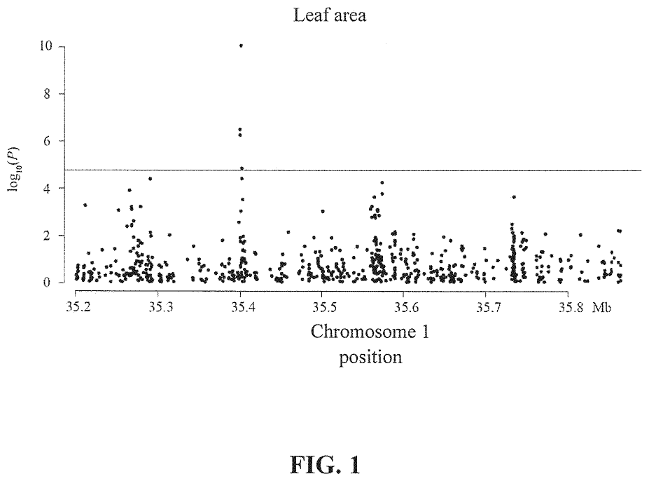

Step 6) the additive and dominant genetic effects of SMPs on leaf size trait are detected. The MLM model is used to perform epigenome-wide association study on the SMPs and leaf area trait under the population structure and kinship matrix. The significantly associated SMPs were identified under the threshold is P<1/n (n is the number of DNA methylation sites, Bonferroni correction). Then the additive and dominant genetic effects are analyzed by the Tassel 5.0 software. The results are shown in FIG. 1. FIG. 1 shows the results of genome-wide epigenetic association analysis of the leaf area (shown in the Manhattan plot), and a specific region on chromosome 1 of Populus tomentosa is shown, which significantly associated DNA methylation sites are shown above the horizontal line. Table 2 shows the additive and dominant genetic effects of the significantly associated SMPs of the leaf area.

TABLE 2
Additive and dominant genetic effects of the
significantly associated SMPs underlying the leaf area

| | | Additive | Dominant |
|---|---|---|---|
| SMP_ID | P_value | effect | effect |
| chr01_35476367 | 0.000000565 | — | 18.61 |
| chr01_35476368 | 0.000000367 | −4.34 | — |
| chr01_35477495 | 0.000000000536 | 5.54 | −2.80 |
| chr01_35478662 | 0.00000741 | 6.88 | — |

### Example 2

Specific operation steps are as follows:

Step 1): The natural population is of 5-year-old, 300 Populus tomentosa genotypic individuals planted in Guanxian County, Shandong, China. The functional leaves (the fourth to sixth leaves from the top of the stem) are collected from 9:00 to 11:00 AM, and in order to prevent changes in its DNA methylation pattern, the functional leaves are immediately frozen in liquid nitrogen (−196° C.) after collection.

Step 2): the genomic DNA of the leaf samples are isolated using DNeasy Plant Mini Kit (Qiagen China, Shanghai, China).

After the foregoing steps are completed, the genomic DNA can be further detected, specifically: 2.1: the degree of degradation of the DNA sample and the RNA contamination are determined by agarose gel electrophoresis; 2.2: the OD260/OD280 ratio of each DNA sample is detected using Nanodrop to determine the purity of the DNA sample; and 2.3: the concentration of each DNA sample is accurately quantified using Qubit2.0 Flurometer (Life Technologies, CA, USA).

Then, the method of performing bisulfite sequencing on the extracted genomic DNA, and constructing the bisulfite-treated DNA library based on the genomic DNA in step 3) uses a conventional technical method. The specific implementation of the present invention is as follows:

Step 3.1: the genomic DNA is randomly fragment to 200-300 bp by using Covaris S220.

Step 3.2: end repairing and tail A addition are performed on the DNA fragments using sequencing adapters in which all cytosines are methylated, the purpose of which is to provide sequence information for the primers required for the PCR amplification.

Step 3.3: the DNA fragments in step 3.2 are twice treat with bisulfite. After the bisulfite treatment, C which is not methylated becomes U (which becomes T after PCR amplification), and the methylated C remains unchanged. Specifically, the bisulfite treatment is carried out using EZ DNA Methylation Gold Kit (Zymo Research, Murphy Ave., Irvine, Calif., U.S.A.).

Step 3.4: the bisulfite-treated DNA fragments in step 3.3 are subjected to PCR amplification to construct a MethylC-seq library.

Step 3.5: sequencing is performed on the MethylC-seq library.

The DNA isolation, MethylC-seq library construction, and sequencing were performed by Beijing Novogene Biological Information Technology Co., Ltd.

Step 4): identifying DNA methylation sites according to a sequencing reads of each sample, and performing genotyping on the SMPs. The sequencing reads of each sample are aligned to the Populus tomentosa reference genome using the Bismark and the Bowtie2 software with default parameters to identify the SMPs. The methylation support rate of each DNA methylation site is calculated for genotyping. Specifically, the methylation support rate (MSR) of the DNA methylation sites in each individual, which is calculated by the formula:

$$DNA methylation support rate ( MSR ) = \frac{methylated reads}{methylated reads + unmethylated reads}$$

if MSR of the site is >0.7, the genotyping is homozygous methylated (M:M); if MSR of the site is between 0.3 and 0.7, the genotyping is heterozygous (U:M); and if MSR of the site is <0.3, the genotyping is homozygous unmethylated (U:U);

Step 5) Measurement of stomatal conductance traits. The functional leaves (the fourth to sixth leaves from the top of the stem) are collected at the same time as the leaf samples for extracting the genomic DNA. Then, the functional leaves of each individuals were used to measuring the stomatal conductance by the LI-6400 portable photosynthesis system (LI-COR Inc., Lincoln, Nebr., USA). The stomatal conductance phenotypic value is shown in Table 3.

TABLE 3
Stomatal conductance trait of 300 genotypic individuals
of a natural population of Populus tomentosa (unit: mol ·
m⁻² · s⁻¹)

| Indiv. | Stomatal |
|---|---|
| No. | conductance |
| P1 | 0.258 |
| P2 | 0.227 |
| P3 | 0.152 |
| P4 | 0.104 |
| P5 | 0.260 |
| P6 | 0.053 |
| P7 | 0.078 |
| P8 | 0.168 |
| P9 | 0.054 |
| P10 | 0.028 |
| P11 | 0.265 |
| P12 | 0.063 |
| P13 | 0.298 |
| P14 | 0.209 |
| P15 | 0.047 |
| P16 | 0.048 |
| P17 | 0.171 |
| P18 | 0.248 |
| P19 | 0.159 |
| P20 | 0.089 |
| P21 | 0.015 |
| P22 | 0.051 |
| P23 | 0.015 |
| P24 | 0.073 |
| P25 | 0.042 |
| P26 | 0.111 |
| P27 | 0.080 |
| P28 | 0.260 |
| P29 | 0.150 |
| P30 | 0.195 |
| P31 | 0.090 |
| P32 | 0.068 |
| P33 | 0.209 |
| P34 | 0.236 |
| P35 | 0.251 |
| P36 | 0.086 |
| P37 | 0.107 |
| P38 | 0.193 |
| P39 | 0.063 |
| P40 | 0.019 |
| P41 | 0.062 |
| P42 | 0.227 |
| P43 | 0.189 |
| P44 | 0.107 |
| P45 | 0.050 |
| P46 | 0.272 |
| P47 | 0.220 |
| P48 | 0.079 |
| P49 | 0.121 |
| P50 | 0.018 |
| P51 | 0.094 |
| P52 | 0.060 |
| P53 | 0.024 |
| P54 | 0.163 |
| P55 | 0.238 |
| P56 | 0.237 |
| P57 | 0.051 |
| P58 | 0.261 |
| P59 | 7.702 |
| P60 | 0.158 |
| P61 | 4.552 |
| P62 | 1.793 |
| P63 | 5.242 |
| P64 | 6.424 |
| P65 | 6.288 |
| P66 | 1.177 |
| P67 | 3.511 |
| P68 | 5.980 |
| P69 | 0.191 |
| P70 | 6.411 |
| P71 | 2.399 |
| P72 | 0.521 |
| P73 | 0.502 |
| P74 | 1.143 |
| P75 | 4.796 |
| P76 | 0.386 |
| P77 | 0.892 |
| P78 | 0.568 |
| P79 | 1.258 |
| P80 | 5.852 |
| P81 | 6.810 |
| P82 | 6.318 |
| P83 | 2.317 |
| P84 | 5.949 |
| P85 | 2.036 |
| P86 | 5.017 |
| P87 | 0.795 |
| P88 | 3.640 |
| P89 | 5.191 |
| P90 | 3.755 |
| P91 | 3.596 |
| P92 | 1.332 |
| P93 | 3.900 |
| P94 | 0.286 |
| P95 | 6.846 |
| P96 | 6.915 |
| P97 | 4.113 |
| P98 | 5.949 |
| P99 | 2.541 |
| P100 | 1.980 |
| P101 | 5.108 |
| P102 | 5.161 |
| P103 | 4.002 |
| P104 | 0.473 |
| P105 | 6.714 |
| P106 | 6.309 |
| P107 | 6.605 |
| P108 | 3.216 |
| P109 | 1.740 |
| P110 | 5.112 |
| P111 | 1.790 |
| P112 | 5.837 |
| P113 | 4.768 |
| P114 | 2.112 |
| P115 | 2.105 |
| P116 | 6.314 |
| P117 | 2.738 |
| P118 | 3.507 |
| P119 | 4.875 |
| P120 | 4.889 |
| P121 | 3.012 |
| P122 | 3.496 |
| P123 | 4.900 |
| P124 | 2.632 |
| P125 | 5.616 |
| P126 | 1.949 |
| P127 | 4.334 |
| P128 | 6.489 |
| P129 | 3.417 |
| P130 | 2.220 |
| P131 | 4.948 |
| P132 | 1.547 |
| P133 | 6.973 |
| P134 | 1.325 |
| P135 | 4.926 |
| P136 | 6.315 |
| P137 | 2.451 |
| P138 | 3.593 |
| P139 | 2.761 |
| P140 | 4.571 |
| P141 | 6.337 |
| P142 | 3.424 |
| P143 | 5.204 |
| P144 | 3.826 |
| P145 | 6.532 |
| P146 | 6.930 |
| P147 | 4.321 |
| P148 | 0.817 |
| P149 | 2.754 |
| P150 | 6.488 |
| P151 | 0.003 |
| P152 | 3.434 |
| P153 | 6.168 |
| P154 | 5.678 |
| P155 | 2.431 |
| P156 | 2.321 |
| P157 | 6.207 |
| P158 | 1.014 |
| P159 | 5.414 |
| P160 | 6.745 |
| P161 | 0.203 |
| P162 | 4.738 |
| P163 | 2.823 |
| P164 | 6.120 |
| P165 | 1.387 |
| P166 | 0.778 |
| P167 | 3.501 |
| P168 | 1.421 |
| P169 | 3.389 |
| P170 | 4.788 |
| P171 | 2.939 |
| P172 | 2.618 |
| P173 | 1.863 |
| P174 | 5.977 |
| P175 | 0.407 |
| P176 | 2.436 |
| P177 | 2.843 |
| P178 | 4.030 |
| P179 | 6.926 |
| P180 | 6.632 |
| P181 | 5.677 |
| P182 | 4.716 |
| P183 | 6.456 |
| P184 | 2.130 |
| P185 | 0.821 |
| P186 | 1.877 |
| P187 | 6.165 |
| P188 | 5.600 |
| P189 | 5.216 |
| P190 | 1.314 |
| P191 | 4.615 |
| P192 | 1.425 |
| P193 | 1.206 |
| P194 | 5.523 |
| P195 | 1.097 |
| P196 | 6.355 |
| P197 | 5.797 |
| P198 | 6.625 |
| P199 | 5.087 |
| P200 | 0.026 |
| P201 | 2.113 |
| P202 | 5.660 |
| P203 | 5.908 |
| P204 | 5.261 |
| P205 | 2.198 |
| P206 | 6.399 |
| P207 | 0.378 |
| P208 | 3.647 |
| P209 | 6.803 |
| P210 | 6.920 |
| P211 | 1.002 |
| P212 | 0.262 |
| P213 | 4.849 |
| P214 | 3.847 |
| P215 | 0.589 |
| P216 | 5.112 |
| P217 | 1.893 |
| P218 | 1.501 |
| P219 | 4.583 |
| P220 | 4.009 |
| P221 | 2.806 |
| P222 | 1.936 |
| P223 | 4.493 |
| P224 | 2.935 |
| P225 | 6.782 |
| P226 | 2.257 |
| P227 | 2.267 |
| P228 | 3.780 |
| P229 | 4.908 |
| P230 | 2.207 |
| P231 | 3.356 |
| P232 | 3.070 |
| P233 | 0.634 |
| P234 | 2.522 |
| P235 | 3.062 |
| P236 | 2.078 |
| P237 | 1.747 |
| P238 | 4.851 |
| P239 | 1.332 |
| P240 | 0.227 |
| P241 | 0.251 |
| P242 | 0.032 |
| P243 | 0.094 |
| P244 | 0.112 |
| P245 | 0.081 |
| P246 | 0.089 |
| P247 | 0.139 |
| P248 | 0.053 |
| P249 | 0.257 |
| P250 | 0.066 |
| P251 | 0.276 |
| P252 | 0.079 |
| P253 | 0.260 |
| P254 | 0.080 |
| P255 | 0.040 |
| P256 | 0.253 |
| P257 | 0.048 |
| P258 | 0.145 |
| P259 | 0.059 |
| P260 | 0.115 |
| P261 | 0.063 |
| P262 | 0.203 |
| P263 | 0.298 |
| P264 | 0.153 |
| P265 | 0.311 |
| P266 | 2.756 |
| P267 | 1.188 |
| P268 | 5.814 |
| P269 | 6.607 |
| P270 | 6.107 |
| P271 | 0.873 |
| P272 | 1.551 |
| P273 | 5.731 |
| P274 | 3.718 |
| P275 | 6.090 |
| P276 | 5.812 |
| P277 | 2.363 |
| P278 | 2.034 |
| P279 | 5.149 |
| P280 | 1.649 |
| P281 | 4.447 |
| P282 | 5.860 |
| P283 | 0.544 |
| P284 | 3.543 |
| P285 | 5.083 |
| P286 | 3.652 |
| P287 | 1.283 |
| P288 | 6.147 |
| P289 | 3.518 |
| P290 | 0.816 |
| P291 | 6.110 |
| P292 | 3.081 |
| P293 | 3.481 |
| P294 | 1.530 |
| P295 | 3.403 |
| P296 | 1.362 |
| P297 | 0.321 |
| P298 | 3.707 |
| P299 | 6.424 |
| P300 | 2.594 |

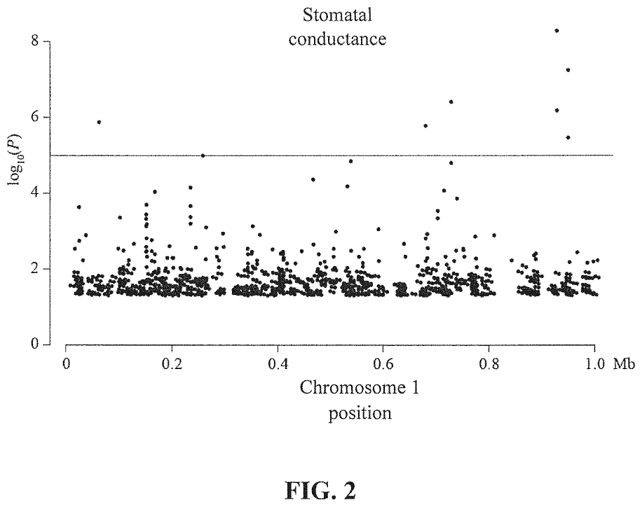

Step 6) the additive and dominant genetic effects of SMPs on stomatal conductance trait are detected. The MLM model is used to perform epigenome-wide association study on the SMPs and stomatal conductance trait under the population structure and kinship matrix. The significantly associated SMPs are identified under the threshold P<1/n (n is the number of DNA methylation sites, Bonferroni correction). Then the additive and dominant genetic effects are analyzed using the Tassel 5.0 software. The results are shown in FIG. 2. FIG. 2 shows the results of genome-wide epigenetic association analysis of the stomatal conductance (shown in the Manhattan plot), and a specific region on chromosome 1 of Populus tomentosa is shown, where significantly associated DNA methylation sites are shown above the horizontal line. Table 4 shows the additive and dominant genetic effects of the significantly associated SMPs of the stomatal conductance.

TABLE 4
Additive and dominant genetic effects of the
significantly associated SMPs underlying
the stomatal conductance

| SMP_ID | P_value | Additive effect | Dominant effect |
|---|---|---|---|
| chr01_928366 | 0.00000000539 | −3.778696064 | −3.760257703 |
| chr01_949225 | 0.0000000571 | — | 7.552436241 |
| chr01_728260 | 0.000000393 | — | 0.094728243 |
| chr01_928367 | 0.000000664 | — | 0.165908058 |
| chr01_63116 | 0.00000136 | — | 0.150602667 |
| chr01_680224 | 0.00000171 | −0.13269173 | — |

As can be seen from the above experimental data, the method provided by the present invention has the advantage of providing the first estimation of the additive and dominant genetic effects of SMPs underlying complex quantitative traits. The present invention provides a scientific theoretical basis for the dissection of the epigenetic architectures of quantitative traits of perennial woody plants, and a new technical guidance for gene marker-assisted breeding, which has important theoretical and technical values.

The foregoing descriptions are only preferred implementation manners of the present invention. It should be noted that for a person of ordinary skill in the field, several improvements and modifications might further be made without departing from the principle of the present invention. These improvements and modifications should also be deemed as falling within the protection scope of the present invention.

What is claimed is:

1. A method for estimating additive and dominant genetic effects of single methylation polymorphisms (SMPs) on quantitative traits, comprising the following steps:
1) collecting the samples of different individuals in natural population at the same stage and same tissue, and isolating the genomic DNA of each sample; measuring the phenotypic data from the individuals in natural population;
2) constructing MethylC-seq libraries using the genomic DNA of each sample in step 1), and performing paired-end sequence to obtain DNA methylation sequencing reads;
3) identifying single methylation polymorphisms (SMPs) from the DNA methylation sequencing reads, and performing genotyping according to the methylation support rate (MSR) of the DNA methylation sites in each individual, which calculated by the formula:
$$DNA methylation support rate ( MSR ) = \frac{methylated reads}{methylated reads + unmethylated reads}$$
if MSR of the site is >0.7, genotyping is homozygous methylated site (M:M); if MSR of the site is between 0.3 and 0.7, genotyping is heterozygous site (U:M); and if MSR of the site is <0.3, genotyping is homozygous unmethylated site (U:U);
4) performing epigenome-wide association study on SMPs obtained in step 3) and the phenotypic data in step 1) by Mixed Linear Model (MLM), and identifying SMPs that were significantly associated with the phenotype;
5) estimating the additive and dominant genetic effects of the significantly associated SMPs using the Tassel 5.0 software package.

2. The method according to claim 1, wherein a threshold for the identifying the significantly associated SMPs in step 4) is P<1/n (Bonferroni correction), where n is the number of SMPs.

3. The method according to claim 1, wherein software for the identifying SMPs, and performing genotyping according to the methylation support rate of the DNA methylation sites in step 3) is Bismark software.

4. The method according to claim 1, wherein the DNA methylation sequencing in step 2) is paired-end sequencing with a read length of 125 bp and a depth of 30×; and the sequencing is performed by Illumina Hiseq 2000/2500 platform.

5. The method according to claim 1, wherein the samples are perennial woody plants.

6. The method according to claim 2, wherein the samples are perennial woody plants.

7. The method according to claim 3, wherein the samples are perennial woody plants.

8. The method according to claim 4, wherein the samples are perennial woody plants.

9. The method according to claim 1, wherein the phenotypic shape comprises leaf area and stomatal conductance.

10. The method according to claim 2, wherein the phenotypic shape comprises leaf area and stomatal conductance.

11. The method according to claim 3, wherein the phenotypic shape comprises leaf area and stomatal conductance.

12. The method according to claim 4, wherein the phenotypic shape comprises leaf area and stomatal conductance.

13. Use of the method according to claim 1 in plant molecular breeding.

14. Use of the method according to claim 2 in plant molecular breeding.

15. Use of the method according to claim 3 in plant molecular breeding.

16. Use of the method according to claim 4 in plant molecular breeding.
